(19) United States
(12) Patent Application Publication
(10) Pub. No.: US 2006/0062756 A1
(43) Pub. Date: Mar. 23, 2006

(54) Method of cancer screening; method of cancer treatment; method of diabetes treatment; method of multiple sclerosis treatment; method of interstitial cystitis treatment; method of acquired immune deficiency syndrome treatment; and method of herpes treatment

(76) Inventor: John R. Woodward, Dallas, TX (US)

Correspondence Address: Michael A. O'Neil;Michael A. O'Neil, P.C., Suite 820, 5949 Sherry Lane, Dallas, TX, 75225, US

(21) Appl. No.: 11/111,422

(22) Filed: Apr. 21, 2005

Related U.S. Application Data
(63) Continuation-in-part of application No. 11/003,293, filed on Dec. 3, 2004.
Application No. 11/003,293 is a continuation of application No. 10/946,213, filed on Sep. 21, 2004.

(51) Int. Cl. A61K 38/19; A61K 31/522; A61K 31/4745

(52) U.S. Cl. 424/85.1; 514/263.32; 514/291

(57) ABSTRACT

A method of cancer screening comprising the steps of administering the Blood CA 27,29 test; administering a mammogram; administering an needle biopsy; administering a PET scan; and administering a blood tumor cell count. If all of the foregoing steps are positive, the cancer is treated by applying imiquimod transdermally, preferably by mixing ALDARA™ 5% cream with an equal amount of H base cream™; administering a vaccine containing tumor necrosis factor alpha, preferably the BCG vaccine; and orally administering Valtrex™ daily. The foregoing treatment method is also effective in treating Type I diabetes, obesity, multiple sclerosis, interstitial cystitis, acquired immune deficiency syndrome, herpes simplex, and herpes zoster.

### CROSS-REFERENCE TO RELATED APPLICATIONS

This application is a continuation-in-part of application Ser. No. 11/003,293 filed Dec. 3, 2004, currently pending, which is a continuation-in-part of application Ser. No. 10/946,213 filed Sep. 21, 2004, currently pending.

### TECHNICAL FIELD

This invention relates generally to the detection and treatment of auto-immune disorders and cancers, and more particularly to a method of screening for and a method of treating duct cell cancer of the breast, squamous cell cancer of the uterine cervix, anal cancer, diabetes, obesity, multiple sclerosis, interstitial cystitis, acquired immune deficiency syndrome (AIDS), herpes simplex, and herpes zoster.

### BACKGROUND AND SUMMARY OF THE INVENTION

As is well known, various technologies are available to the medical profession for use in determining the presence of cancers in patients. Included are x-ray studies, magnetic resonance imaging (MRI) studies, CT scans, as well as studies of various body fluids such as blood, urine, etc. Potential sites for colon cancer, for example, can be investigated utilizing electro-optical technologies. In some cases needle biopsy or exploratory surgery is necessary to confirm either the presence or absence of suspected cancer.

Various techniques for treating cancers are also well known. Certain cancers can be surgically removed, whereas other cancers require radiation therapy, chemotherapy, or combinations of radiation therapy and chemotherapy. Other cancers are susceptible to control using one or more drug therapies.

Type I diabetes is generally diagnosed in juveniles and young adults. In type I diabetes, the pancreas does not make insulin, which is necessary for the body to process sugars. Persons with Type I diabetes can live long, healthy lives, but must be careful with their diet and must take insulin to manage their blood glucose levels. Currently, the only treatment for Type I diabetes is to take insulin, or receive pancreas or islet cell transplants.

Obesity is a disease that affects persons of all ages. As opposed to simply being overweight, obesity is not the result of over-eating. Rather, obesity is a caused by a viral infection, specifically the Coxsackie virus, which reduces an individual's overall well-being, can result in a person developing diabetes, increases the risk of disabilities linked to mental illness and musculoskeletal problems, and in some cases may shorten a person's lifespan. Heretofore there have been many treatments for obesity including but not limited to gastric and/or intestinal bypass surgery, numerous prescription medications, hormone treatments, and strict diet and exercise regimes. Surgery has been proven effective but is very costly. Prescription medications and hormone treatments are not always effective and can be very costly. Diet and exercise can be effective for some persons, but for persons with extreme obesity, diet and exercise are not enough. Further, for most persons, diet and exercise require discipline and patience in order for the treatment to be effective.

Multiple sclerosis (MS) is a chronic, unpredictable disease of the nervous system that afflicts over 2.5 million persons worldwide. An MS attack destroys myelin, the protective fibers around nerve fibers in the central nervous system. The destroyed myelin is replaced by scars of hardened “sclerotic” tissue, and some nerve endings are permanently severed. The common symptoms are loss of balance, fatigue, poor circulation, slurred speech, blindness, and in some cases paralysis. Currently, the only treatment is disease-modifying drugs, including drugs with a chemotherapeutic agent. These treatments only modify the disease to lessen the severity or frequency of the MS attacks.

Interstitial cystitis is an auto-immune disease similar to diabetes where the body attacks its own cells, specifically the urinary bladder. Persons suffering from interstitial cystitis suffer from constant suprapubic pain brought on by bladder distention, causing a constant urge to urinate. Heretofore the only treatments for interstitial cystitis have been prescription medication to control the bladder pains and urges to urinate but no cure is currently available.

Acquired immune deficiency syndrome (AIDS) is the most serious stage of the human immunodeficiency virus (HIV) and is a fatal disease. AIDS attacks and destroys the disease-fighting cells of the immune system, leaving the body with a weakened defense against opportunistic and life-threatening diseases such as pneumonia, cancer, nervous system diseases, and various other bacterial diseases and infections. AIDS and HIV are managed by medications and monitoring a patient's viral load, specifically testing to determine the patient's count of the number of CD4 cells in a blood sample. CD4 cells, also called T cells or CD4⁺ T cells, are white blood cells that fight infection. HIV destroys CD4 cells, making it harder for the body to fight infections.

The most recommended treatment for HIV infections is Highly Active Antiretroviral Therapy (HAART) which combines three or more anti-HIV medications in a daily regimen. Heretofore treatments available for HIV and AIDS have been unable to cure HIV infections but rather to control the symptoms of HIV. Further, these treatments are very high in cost and therefore difficult to obtain and/or unavailable to persons of low income or persons without health care insurance.

Herpes simplex affects a significant amount of the American adult population in one or more of its various forms. For example, oral herpes, an infection caused by the herpes simplex virus, is estimated to be present in 50 to 80 percent of the American adult population, and 20 percent are infected with genital herpes, also caused by the herpes simplex virus. To date, there is no cure for herpes. The primary treatments are suppressive antiviral therapy.

Herpes zoster, commonly known as shingles, is caused by the same virus responsible for chicken pox and can cause a wide range of problems affecting the skin and the eye. Heretofore, the treatments available for herpes zoster work to treat the symptoms of the disease, but do not eliminate the herpes altogether. As a result, herpes zoster lies dormant in certain nerve fibers after initial exposure. Herpes zoster may become active as a result of many factors including aging, stress, suppression of the immune system, and certain medications. In some cases, the serious secondary conditions caused by herpes zoster may require surgery.

The present invention comprises a method of cancer screening, a method of cancer treatment, a method of diabetes treatment, a method of obesity treatment, a method of multiple sclerosis treatment, a method of interstitial cystitis treatment, a method of acquired immune deficiency syndrome (AIDS) treatment, a method of herpes simplex treatment, and a method of herpes zoster treatment which have proven successful in controlling epidermal cancers including, but not limited to, duct cell breast cancer, cervical squamous cancer, anal cancer, and in controlling Type I diabetes and multiple sclerosis. In accordance with the broader aspects of the invention, a method of cancer screening involves a series of testing procedures each more expensive than the one before. Only when results of each of the testing procedures is positive is the presence of cancer confirmed. The invention further comprises a method of treating cancer, insulin dependent Type I diabetes, obesity, multiple sclerosis, interstitial cystitis, acquired immune deficiency syndrome (AIDS), herpes simplex, and herpes zoster wherein the drug imiquimod is administered transdermally in conjunction with a vaccine containing tumor necrosis factor, for example the BCG vaccine, and valacyclovia hydrochlorine tablets. The method of the present invention treats Type I diabetes and obesity by enabling the body to regenerate islet cells. Multiple sclerosis is managed and treated by enabling the body to repair nerve endings and regenerate damaged fibers. Interstitial cystitis is treated and possibly cured by enabling the body to regenerate its own cells.

AIDS is treated by increasing the T cell or CD4 count in a patient's blood, strengthening the patient's immune system and thereby transforming AIDS from a fatal disease to a manageable chronic disease. Herpes simplex and herpes zoster are also treated by increasing the T cell count in the patient's blood so that the imiquimod is able to attack and kill the virus and prevent the infected cells from replicating.

The treatment method of the present invention is relatively low in cost and therefore may be made available to more patients and likewise reduce the rising healthcare costs that have long been associated with treating the aforementioned chronic and fatal diseases.

### BRIEF DESCRIPTION OF THE DRAWING

A more complete understanding of the present invention may be had by reference to the following Detailed Description when taken in connection with the accompanying Drawings, wherein:

FIG. 1 is a flowchart illustrating initial steps in the cancer screening method of the present invention;

FIG. 2 is a flowchart illustrating subsequent steps in the cancer screening method of the present invention; and

FIG. 3 is a flowchart illustrating the diabetes treatment method of the present invention.

### DETAILED DESCRIPTION

### Introduction

The following examples describe a method of detecting and treating duct cell breast cancer, and a method for treating Type I diabetes. However, the present invention is equally applicable to other epidermal cancers, such as squamous cancer of the uterine cervix and anal cancer, the treatment and management of obesity, the treatment and management of multiple sclerosis, the treatment and cure of interstitial cystitis, the treatment and management of AIDS, and the treatment and management of herpes simplex and herpes zoster.

### EXAMPLE

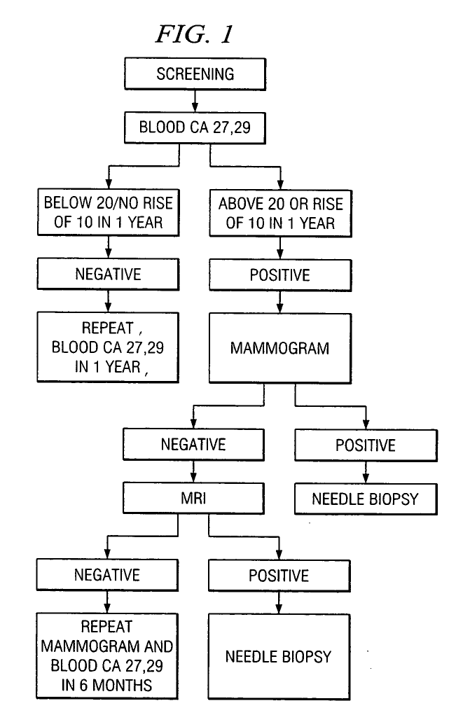

Referring to the Drawings, and particularly to FIG. 1 thereof, the early steps in the method of cancer screening of the present invention are shown therein. Screening begins with administration of the testing procedure known as Blood CA 27,29. The Blood CA 27,29 testing procedure has heretofore been utilized in monitoring the results of existing cancer treatment procedures. However, the Blood CA 27,29 procedure has not heretofore been used for cancer screening.

If the number comprising the results of the Blood CA 27,29 procedure is less than 20, and if there has been no increase in the number comprising the result of the Blood CA 27,29 testing procedure of ten (10) or more in the immediately preceding year, the result of the Blood CA 27,29 testing procedure is considered to be negative. The patient is then scheduled for follow-up testing utilizing the Blood CA 27,29 procedure in one year.

If the number comprising the result of the Blood CA 27,29 procedure is 20 or above, or if there has been an increase of 10 or more in the number comprising the result of the CA 27,29 testing procedure in the immediately preceding year, the result of the Blood CA 27,29 procedure is considered to be positive. In that event a mammogram testing procedure is administered. If the result of the mammogram testing procedure is negative, an MRI testing procedure is administered. If the result of the MRI testing procedure is negative, both the mammogram testing procedure and the Blood CA 27,29 testing procedure are re-administered in six months time. Conversely, if either the mammogram testing procedure is positive or the MRI testing procedure is positive, a needle biopsy of the identified lesion is performed.

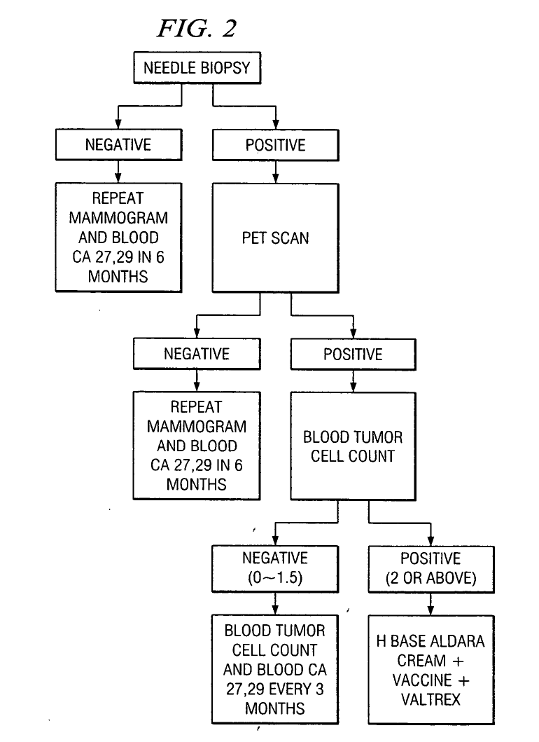

Referring to FIG. 2, if the results of the needle biopsy testing procedure are negative, both the mammogram testing procedure and the Blood CA 27,29 testing procedure are re-administered in six months. If the needle biopsy testing procedure is positive, a positron emission tomography (PET) scan testing procedure is administered. If the result of the PET scan testing procedure is negative, the mammogram testing procedure and the Blood CA 27,29 testing procedure are re-administered in six months. If the result of the PET scan testing procedure is positive, a blood tumor cell count testing procedure is administered. If the result of the blood tumor cell count testing procedure is negative, that is, if the number comprising the result of the blood tumor cell count testing procedure is between 0 and 1.5, the blood tumor cell count testing procedure and the Blood CA 27,29 testing procedure are administered at three month intervals. Conversely, if the blood tumor cell count testing procedure is positive, that is, if the number comprising the result of the blood tumor cell count testing procedure is two or above, the cancer treatment procedure of the present invention is administered.

The cancer treatment procedure of the present invention comprises the transdermal administration of the drug imiquimod combined with a vaccine containing tumor necrosis factor and administration of valacyclovia hydrochlorine tablets. Imiquimod is commercially available from 3M Pharmaceuticals under the trademark ALDARA™. A healthy human body produces the protein interferon alpha in response to an infection. Interferon alpha works to coat the infection or virus in order to make the infection or virus vulnerable to the human immune system. Imiquimod, i.e. ALDARA™ cream, turns into interferon alpha once inside the human body, providing the needed interferon alpha not adequately produced by the patient's own body. In accordance with the present invention, ALDARA™ cream 5% is mixed at a 1:1 ratio with H base cream. The ingredients of H base cream are:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.
   The mixture of imiquimod and H base cream as described above is administered transdermally, preferably by mixing ¼ cc ALDARA™ 5% cream with ¼ cc H base cream and applying the resulting mixture to various locations, i.e., the inner thigh, abdomen, hip, arms, etc., of the patient. Various sites of administration prevent any possible skin irritation. The foregoing amount of the mixture of ALDARA™ 5% cream and H base cream is applied daily.

Tumor necrosis factor (also called TNFa, cachexin, or cachetin) stimulates T-cells that coordinate the immune system. In a healthy human body, tumor necrosis factor is released by white blood cells and other tissues in response to damage caused by an infection. Tumor necrosis factor is found in several vaccines. The preferable vaccine to be used in accordance with the present invention is the BCG vaccine, which is a common vaccine for tuberculosis, given in the United States and around the world. The tumor necrosis factor also known as cachetin included in the BCG vaccine and imiquimod, i.e. ALDARA™ cream, work together to stimulate and reprogram the T-cells to attack the virus coated with interferon alpha. Another source of tumor necrosis factor is Dehydroepiandrosterone (DHEA) but the route of administration of tumor necrosis factor from DHEA must be non-oral. Topical H base cream is ideal for both tumor necrosis factor and interferon alpha administration.

In accordance with the present invention, the vaccine is given in doses of 0.1 mL (100 μg) once every three weeks as long as the treatment continues. Valacyclovia hydrochlorine tablets, available from GlaxoSmithKline under the trademark Valtrex™ is drug commonly used in the treatment for genital herpes. In accordance with the present invention, 1000 mg of Valtrex™ tablets are consumed daily either in one dose of 1000 mg or two doses of 500 mg each. The combination of the ALDARA™ and H base cream, the BCG vaccination, and the Valtrex™ tablets is administered until a blood tumor cell count indicates that there are no cancer cells in the blood and a subsequent blood tumor cell count verifies a normal cell count and no mestastases are present. The results of the procedure are periodically monitored utilizing the Blood CA 27,29 testing procedure.

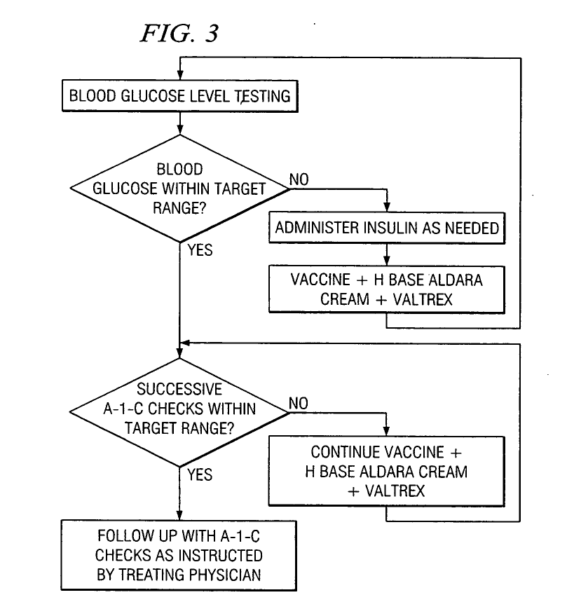

Referring to FIG. 3 thereof, persons with Type I diabetes must check their blood glucose levels at multiple intervals as directed by their physician. The average fasting blood glucose level should be between 70 mg/dL and 110 mg/dL. If the blood glucose level is not within the target level, the level must be corrected by taking insulin.

The diabetes treatment procedure of the present invention comprises the transdermal administration of the drug imiquimod combined with a BCG vaccination and administration of valacyclovia hydrochlorine tablets. Imiquimod is commercially available from 3M Pharmaceuticals under the trademark ALDARA™. A healthy human body produces the protein interferon alpha in response to an infection. Interferon alpha works to coat the infection or virus in order to make the infection or virus vulnerable to the human immune system. Imiquimod, i.e. ALDARA™ cream, turns into interferon alpha once inside the human body, providing the needed interferon alpha not adequately produced by the patient's own body.

In accordance with the present invention, ALDARA™ cream 5% is mixed at a 1:1 ratio with H base cream. The ingredients of H base cream are:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.
   The mixture of imiquimod and H base cream as described above is administered transdermally, preferably by mixing ¼ cc ALDARA™ 5% cream with ¼ cc H base cream and applying the resulting mixture to various locations, i.e., the inner thigh, abdomen, hip, arms, etc., of the patient. Various sites of administration prevent any possible skin irritation. The foregoing amount of the mixture of ALDARA™ 5% cream and H base cream is applied daily.

Tumor necrosis factor (also called TNFa, cachexin, or cachetin) stimulates T-cells that coordinate the immune system. In a healthy human body, tumor necrosis factor is released by white blood cells and other tissues in response to damage caused by an infection. Tumor necrosis factor is found in several vaccines. The preferable vaccine to be used in accordance with the present invention is the BCG vaccine, which is a common vaccine for tuberculosis, given in the United States and around the world. The tumor necrosis factor also known as cachetin included in the BCG vaccine and imiquimod, i.e. ALDARA™ cream, work together to stimulate and reprogram the T-cells to attack the virus coated with interferon alpha. Another source of tumor necrosis factor is Dehydroepiandrosterone (DHEA) but the route of administration of tumor necrosis factor from DHEA must be non-oral. Topical H base cream is ideal for both tumor necrosis factor and interferon alpha administration.

In accordance with the present invention, the vaccine is given in doses of 0.1 mL (100 μg) once every three weeks as long as the treatment continues. Valacyclovia hydrochlorine tablets, available from GlaxoSmithKline under the trademark Valtrex™ is drug commonly used in the treatment for genital herpes. In accordance with the present invention, 1000 mg of Valtrex™ tablets are consumed daily either in one dose of 1000 mg or two doses of 500 mg each.

The results of the procedure are monitored utilizing blood sugar meters and a diary to record ongoing blood sugar levels. Additionally, A-1-C checks track the patient's overall blood sugar levels over two to three month periods, and is the most effective way to track long-range success of the treatment. The treatment method regenerates islet cells, which produce insulin. Once the patient no longer depends on insulin to correct blood sugar levels, the treatment continues until the patient has two or more sequential A-1-C checks in the target range, depending on the judgment of the treating physician. Once treatment is discontinued, A-1-C checks continue, but at less frequent intervals as recommended by the treating physician.

The obesity treatment procedure of the present invention follows the same course of treatment as the Type I diabetes treatment diagramed in FIG. 3 and described hereinabove in conjunction therewith.

The interstitial cystitis treatment procedure of the present invention follows the same course of treatment as the cancer treatment diagramed in FIG. 2 and the diabetes treatment diagramed in FIG. 3 and described hereinabove in conjunction therewith. The interstitial cystitis treatment procedure of the present invention differs from the cancer treatment and the diabetes treatment in that there are currently no tests to monitor or diagnose interstitial cystitis. The method for gauging the effectiveness of the treatment comprises monitoring the frequency and severity of the patient's pain and sensation levels as the treatment course progresses.

The AIDS treatment procedure of the present invention follows the same course of treatment as the cancer treatment diagramed in FIG. 2 and the diabetes treatment diagramed in FIG. 3 and described hereinabove in conjunction therewith. The AIDS treatment procedure of the present invention differs from the cancer treatment and the diabetes treatment in that the effectiveness of the treatment is monitored by blood tests to track the number of CD4 cells in the patient's blood. As the CD4 cells increase, the diseased cells are unable to duplicate; therefore with continued treatment the disease becomes a manageable chronic disease rather than a fatal disease.

The herpes simplex and herpes zoster treatment procedures of the present invention follows the same course of treatment as the cancer treatment diagramed in FIG. 2 and the diabetes treatment diagramed in FIG. 3 and described hereinabove and in conjunction therewith. The herpes simplex and herpes zoster treatment procedures of the present invention differ from the cancer treatment and the diabetes treatment in that the effectiveness of the treatment is monitored by blood tests to track the number of CD4 cells in the patient's blood. As the CD4 cells increase, the diseased cells are unable to duplicate and are eventually killed altogether.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of treating obesity comprising the steps of:
providing a quantity of ALDARA™ (imiquimod)™ 5% cream;
providing a quantity of H base cream™;
mixing ¼ cc. of the ALDARA™ (imiquimod) 5% cream with ¼ cc. of the H base cream™ and transdermally administering the resulting mixture to the patient;
administering a vaccine containing tumor necrosis factor alpha; and
orally administering VALTREX™ (valacyclovir) tablets.

2. The method of treating obesity according to claim 1 wherein the ALDARA™ (imiquimod) 5% cream/H base cream mixture is applied daily, the vaccine containing tumor necrosis factor alpha is administered every three weeks and 500 mg VALTREX™ (valacyclovir) tablets are orally administered twice daily.

3. The method of treating obesity according to claim 1 wherein the ALDARA™ (imiquimod) 5% cream/H base cream mixture is applied daily, the vaccine containing tumor necrosis factor alpha is administered every three weeks and 1000 mg of VALTREX™ (valacyclovir) tablets are orally administered once daily.

4. A method of treating interstitial cystitis comprising the steps of:
providing a quantity of ALDARA™ (imiquimod) 5% cream;
providing a quantity of H base cream™;
mixing ¼ cc. of the ALDARA™ (imiquimod) 5% cream with ¼ cc. of the H base cream™ and transdermally administering the resulting mixture to the patient;
administering a vaccine containing tumor necrosis factor alpha; and
orally administering VALTREX™ (valacyclovir) tablets.

5. The method of treating interstitial cystitis according to claim 4 wherein the ALDARA™ (imiquimod) 5% cream/H base cream mixture is applied daily, the vaccine containing tumor necrosis factor alpha is administered every three weeks, and 500 mg VALTREX™ (valacyclovir) tablets are orally administered twice daily.

6. The method of treating interstitial cystitis according to claim 4 wherein the ALDARA™ (imiquimod) 5% cream/H base cream mixture is applied daily, the vaccine containing tumor necrosis factor alpha is administered every three weeks, and 1000 mg of VALTREX™ (valacyclovir) tablets are orally administered once daily.

7. A method of treating acquired immune deficiency syndrome comprising the steps of:
providing a quantity of ALDARA™ (imiquimod) 5% cream;
providing a quantity of H base cream™;
mixing ¼ cc. of the ALDARA™ (imiquimod) 5% cream with ¼ cc. of the H base cream™ and transdermally administering the resulting mixture to the patient;
administering a vaccine containing tumor necrosis factor alpha; and
orally administering VALTREX™ (valacyclovir) tablets.

8. The method of treating acquired immune deficiency syndrome according to claim 7 wherein the ALDARA™ (imiquimod) 5% cream/H base cream mixture is applied daily, the vaccine containing tumor necrosis factor alpha is administered every three weeks, and 500 mg VALTREX™ (valacyclovir) tablets are orally administered twice daily.

9. The method of treating acquired immune deficiency syndrome according to claim 7 wherein the ALDARA™ (imiquimod) 5% cream/H base cream mixture is applied daily, the vaccine containing tumor necrosis factor alpha is administered every three weeks, and 1000 mg of VALTREX™ (valacyclovir) tablets are orally administered once daily.

10. A method of treating herpes simplex comprising the steps of:
providing a quantity of ALDARA™ (imiquimod) 5% cream;
providing a quantity of H base cream™;
mixing ¼ cc. of the ALDARA™ (imiquimod) 5% cream with ¼ cc. of the H base cream™ and transdermally administering the resulting mixture to the patient;
administering a vaccine containing tumor necrosis factor alpha; and
orally administering VALTREX™ (valacyclovir) tablets.

11. The method of treating herpes simplex according to claim 10 wherein the ALDARA™ (imiquimod) 5% cream/h base cream mixture is applied daily, the vaccine containing tumor necrosis factor alpha is administered every three weeks, and 500 mg VALTREX™ (valacyclovir) tablets are orally administered twice daily.

12. The method of treating herpes simplex according to claim 10 wherein the ALDARA™ (imiquimod) 5% cream/H base cream mixture is applied daily, the vaccine containing tumor necrosis factor alpha is administered every three weeks, and 1000 mg of VALTREX™ (valacyclovir) tablets are orally administered once daily.

13. A method of treating herpes simplex comprising the steps of:
providing a quantity of ALDARA™ (imiquimod) 5% cream;
providing a quantity of H base cream™;
mixing ¼ cc. of the ALDARA™ (imiquimod) 5% cream with ¼ cc. of the H base cream™ and transdermally administering the resulting mixture to the patient;
administering a vaccine containing tumor necrosis factor alpha; and
orally administering VALTREX™ (valacyclovir) tablets.

14. The method of treating herpes simplex according to claim 13 wherein the ALDARA™ (imiquimod) 5% cream/H base cream mixture is applied daily, the vaccine containing tumor necrosis factor alpha is administered every three weeks, and 500 mg VALTREX™ (valacyclovir) tablets are orally administered twice daily.

15. The method of treating herpes simplex according to claim 13 wherein the ALDARA™ (imiquimod) 5% cream/H base cream mixture is applied daily, the vaccine containing tumor necrosis factor alpha is administered every three weeks, and 1000 mg of VALTREX™ (valacyclovir) tablets are orally administered once daily.
